(19) United States
(12) Patent Application Publication
(10) Pub. No.: US 2015/0196621 A1
(43) Pub. Date: Jul. 16, 2015

(54) COMPOSITIONS FOR THE PREVENTION AND TREATMENT OF NEUROINJURY AND METHODS OF USE THEREOF

(71) Applicant: BRAIN-GEN BIOTECH, Duluth, GA (US)

(72) Inventor: Byron D. FORD, Duluth, GA (US)

(21) Appl. No.: 14/540,728

(22) Filed: Nov. 13, 2014

Related U.S. Application Data
(63) Continuation of application No. 12/453,778, filed on May 21, 2009, now abandoned.
(60) Provisional application No. 61/071,901, filed on May 23, 2008.

(51) Int. Cl. A61K 38/18; A61K 31/713; A61K 9/00

(52) U.S. Cl. CPC A61K 38/1883; A61K 9/0019; A61K 31/713

(57) ABSTRACT

A method for preventing or ameliorating secondary neuronal injury and inflammation following traumatic brain injury (TBI) is disclosed. The method comprises the step of administering into a subject in need of such treatment an effective amount of a pharmaceutical composition containing a neuregulin (NRG), a variant of NRG, or an expression vector encoding a NRG or a variant of NRG.

This application is a Continuation of U.S. application Ser. No. 12/453,778, filed May 21, 2009, which claims priority from U.S. Provisional Application Ser. No. 61/071,901, filed on May 23, 2008. The entirety of the aforementioned applications is incorporated herein by reference.

### FIELD

The present invention relates generally to medical treatments and, in particular, to compositions and methods for preventing and treating neuroinjuries, such as acute CNS injuries, with neuregulin (NRG).

### BACKGROUND

Traumatic brain injury (TBI) is a leading cause of morbidity and death in both industrialized and developing countries. TBI is a major and increasing cause of long-term disability in individuals surviving head injuries sustained in military combat. TBI can result from a closed head injury or a penetrating head injury. The incidence of all closed head injuries admitted to hospitals is conservatively estimated to be 200 per 100,000 populations in the United States. The incidence of penetrating head injury in the United States is estimated to be 12 per 100,000, the highest of any developed country in the world. Acute neuronal injury following TBI results in the rapid necrosis of neuronal tissue at the site of injury [Werner, C. et al., Br J Anaesth, 2007. 99(1): p. 4-9]. This primary injury is exacerbated in the ensuing hours and days via the progression of secondary injury mechanism(s) leading to significant neurological dysfunction [Xiong, Y., et al., Biochem Biophys Res Commun, 2001. 286(2): p. 401-5; Xiong, Y., et al., J Neurotrauma, 1998. 15(7): p. 531-44; Sullivan, P. G., et al., Exp Neurol, 2000. 161(2): p. 631-7; Sullivan, P. G., et al., Neuroscience, 2000. 101(2): p. 289-95.] The delayed progression of deterioration of neuronal tissues gives hope that a clinical intervention can be applied in a realistic timeframe following the initial trauma. TBI is characterized by neuroinflammatory pathological sequelae which contribute to brain edema and delayed neuronal cell death [Schumacher, M., et al., Pharmacol Ther, 2007. 116(1): p. 77-106; Stein, S. C., et al., Neurocrit Care, 2004. 1(4): p. 479-88.] To date, there is no targeted pharmacological treatment that effectively limits the progression of secondary injury.

### SUMMARY

One aspect of the present invention relates to a method for preventing or ameliorating secondary neuronal injury and inflammation following traumatic brain injury (TBI). The method comprises the step of administering into a subject in need of such treatment an effective amount of a pharmaceutical composition containing a NRG, a variant of NRG, or an expression vector encoding a NRG or a variant of NRG.

In one embodiment, the method comprises administering into the subject an effective amount of a pharmaceutical composition containing (1) a NRG or a variant of NRG, and (2) an expression vector encoding a NRG or a variant of NRG.

Another aspect of the present invention relates to a method for preventing and treating acute CNS injuries. The method comprises the step of administering into a subject in need of such treatment an effective amount of a pharmaceutical composition containing a NRG, a variant of NRG, or an expression vector encoding a NRG or a variant of NRG.

Yet another aspect of the present invention relates to a kit for preventing or ameliorating secondary neuronal injury and inflammation following traumatic brain injury (TBI). The kit contains (1) a NRG, a variant of NRG, or an expression vector encoding a NRG or a variant of NRG, and (2) an instruction on how to use the NRG, the variant of NRG or the expression vector.

### DETAILED DESCRIPTION OF THE DRAWINGS

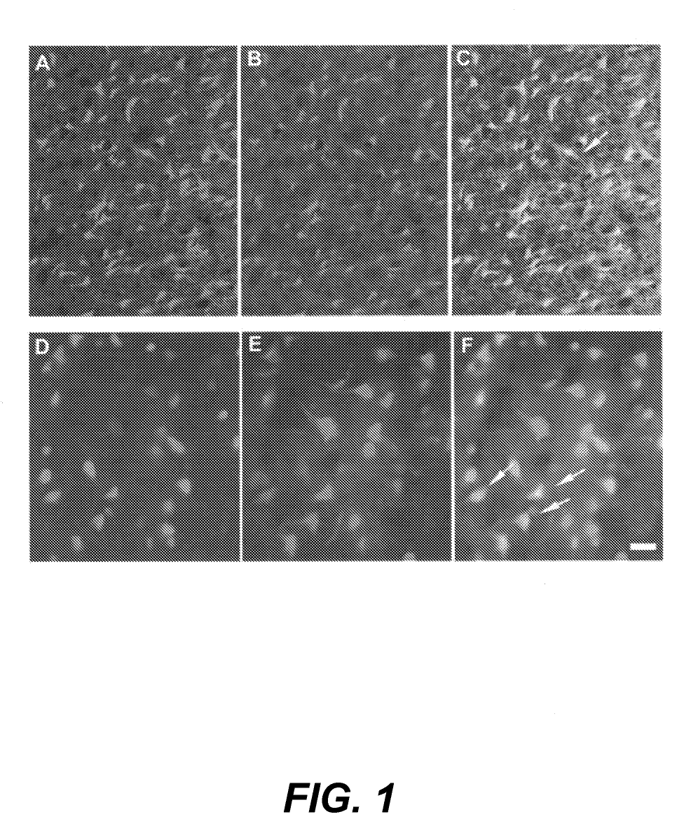

FIG. 1 is a composite of pictures showing ErbB4 receptor expression in apoptotic and degenerating neurons. After ErbB4 immunohistochemistry, brain sections from rats MCAO were stained with Fluoro-Jade, a marker for degenerating neurons. Many neurons in the cortex were Fluoro-Jade-positive (A; green). ErbB4 positive cells (B; red) were co-localized in Fluoro-Jade-positive neurons (C; yellow). Similarly, TUNEL staining (D; green) and erbB4 (E; red) were double-labeled (F; yellow) in a subpopulation of cells in the ipsilateral brain. Arrows indicate examples of double-labeled cells. Scale bar is 40 μM in panels A-C and 20 μM in panels D-F.

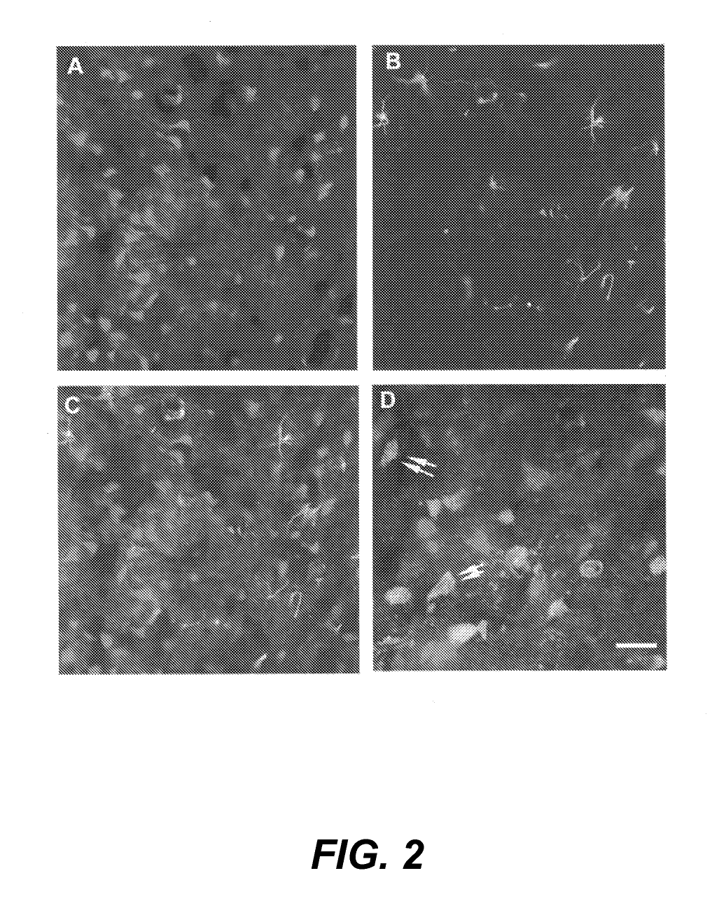

FIG. 2 is a composite of pictures showing erbB4 expression in macrophages/microglia but not astrocytes following MCAO. Sections from the ipsilateral hemisphere were double labeled with antibodies against erbB4 (panel A) and GFAP (panel B). Cells in the peri-infarct regions did not show co-localization of erbB4 and GFAP (panel C). Co-localization of erbB4 (green) and Mac-1/CD11b (red) indicated that erbB4 is found in a subset of macrophages/microglia (panel D). Double arrows indicate examples of double labeled cells). Scale bar is 40 μM in panel A-C and 20 μM in panel D.

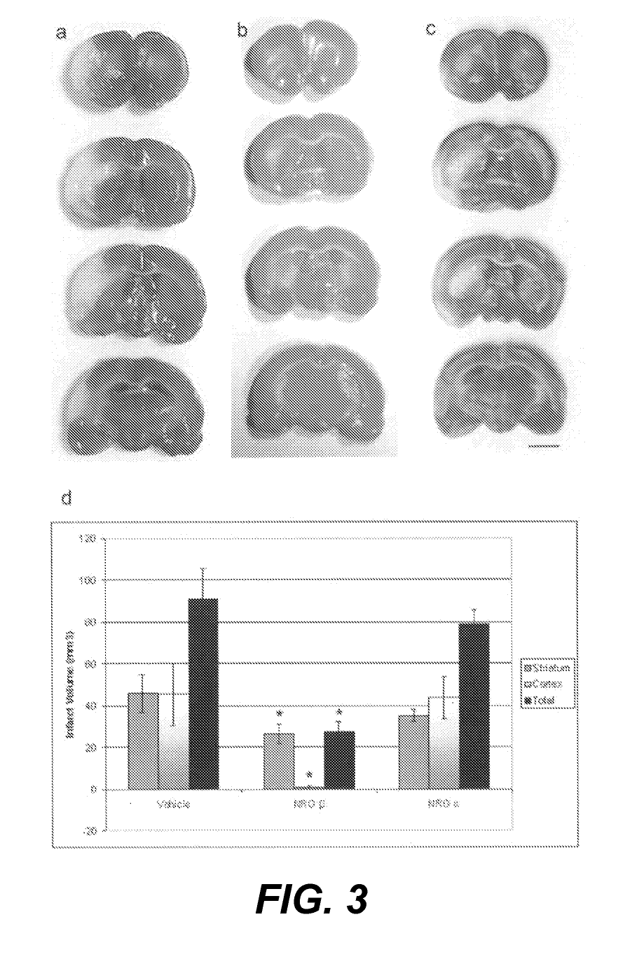

FIG. 3 is a composite of pictures and graphs showing that NRG1β treatment reduce MCAO/reperfusion-induced brain infarction. Representative 2,3,5-triphenyltetrazolium chloride (TTC) stained brain sections are shown from rats injected with vehicle (panel a; n=11), NRG-1α (panel b; n=7) or NRG-1β (panel c; n=3) before MCAO. Infarct volumes in brains from vehicle and NRG-1 treated animals are shown in the graph (panel d). Values are presented as mean±SEM; * denotes significantly different from respective vehicle treated animals (P<0.01).

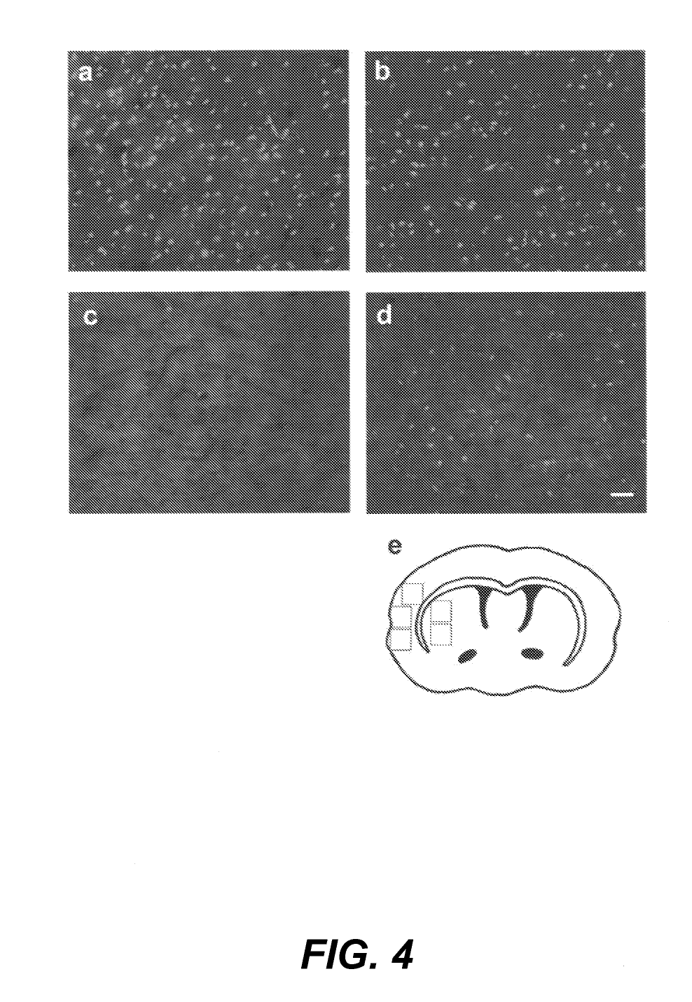

FIG. 4 is a composite of pictures showing that NRG1β suppresses MCAO/reperfusion-induced apoptotic damage in rat brain. Rats were subjected to MCAO for 1.5 hours followed by reperfusion for 24 hours (representative views are shown for TUNEL labeling of rat brain sections; n=5 for each condition). TUNEL staining is found in the cortex (panel a) and striatum (panel b) following MCAO while no TUNEL staining is seen in the cortex (panel c) and reduced levels are seen in the striatum (panel d) in NRG1β-treated rats. The coronal brain image (˜bregma+1.2 mm) indicates the areas observed in the sections (panel e). Scale bar is 100 μM.

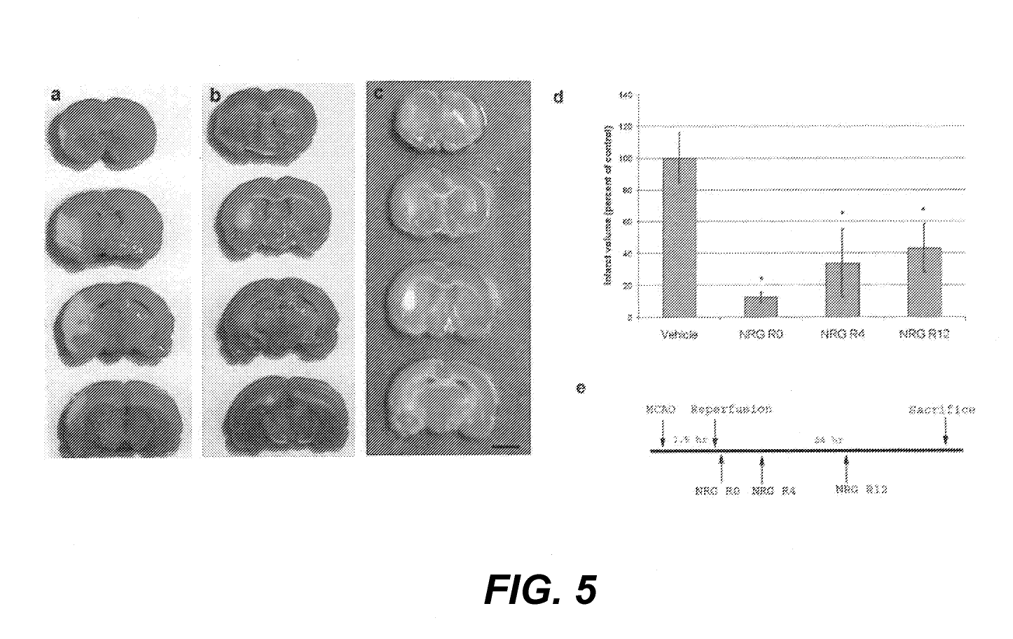

FIG. 5 is a composite of pictures and graphs showing that NRG1 treatment reduces MCAO/reperfusion-induced brain infarction. Representative TTC stained coronal brain sections are shown where rats were injected with vehicle (panel a) or NRG1 immediately after MCAO (panel b) and 4 hours after reperfusion (panel c). Infarct volumes in brains from rats treated with vehicle (n=10) or NRG1 immediately after MCAO (R0; n=8), 4 hours after reperfusion (R4; n=6) or 12 hours after reperfusion (R12; n=8) are show in the graph (panel d). Values are presented as mean±SD of all infarct volumes for each experimental condition; * denotes significantly different from respective vehicle treated animals (P<0.01). The time line (panel e) illustrates the MCAO protocol and NRG1 injections.

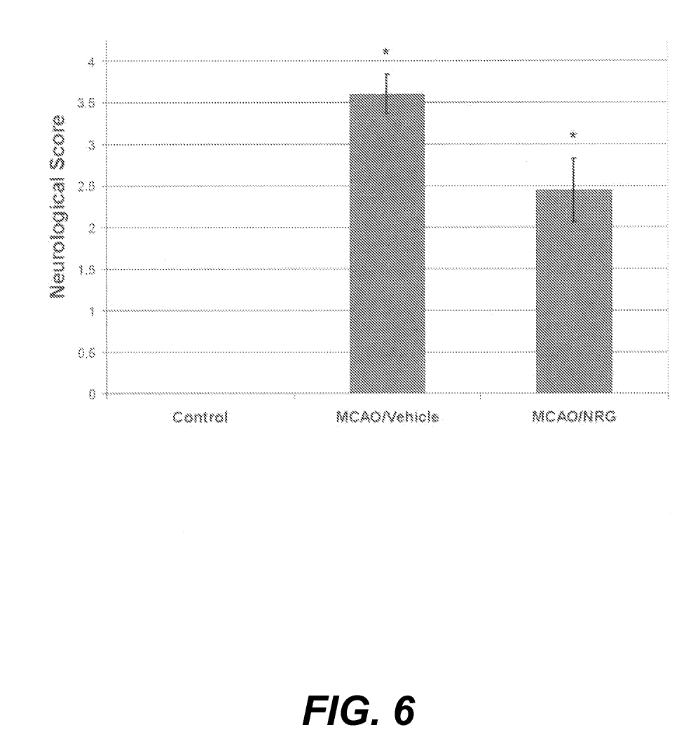

FIG. 6 is a graph showing that NRG1 administration resulted in a significant improvement in neurological outcome (* denotes P<0.01). NRG1 was administered after MCAO and 4 hours of reperfusion. Neurological function was graded on a scale of 0-4 (normal score 0, maximal deficit score 4). All animals were tested prior to surgery (controls; n=14) and after treatment with NRG1 or vehicle. The NRG1 treated group (n=9) displayed a 33% improvement in neurological score compared with vehicle treated rats (n=5).

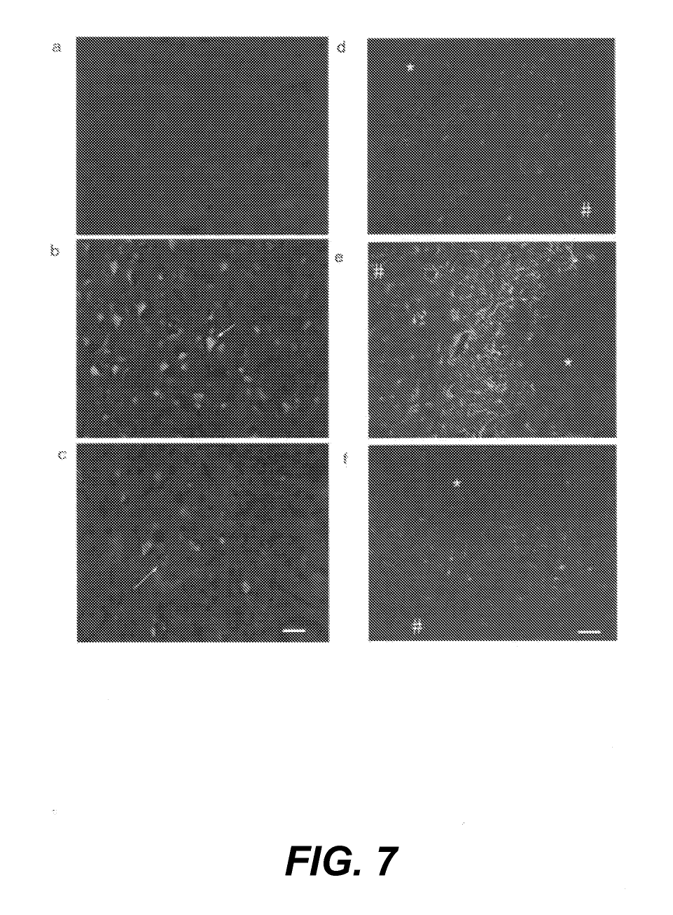

FIG. 7 is a composite showing that NRG1β prevents microglial and astrocytic activation following MCAO. Rats were subjected to MCAO followed by reperfusion for 24 hours (n=5 for each condition). NRG1β or vehicle was injected into the ECA. Sections were labeled for immunohistochemistry with an antibody against ED-1. While no staining was seen in the contralateral side (panel a), ED-1 labeled cells are present in the ipsilateral hemisphere (panel b) following MCAO in vehicle-treated animals. Few ED-I positive cells are found in animals treated with NRG-1β (panel c). Examples of ED-1 positive cells are indicated by the arrows. Scale bar is 50 μM. To assess astrocytic activation, sections were labeled for immunohistochemistry with an antibody against GFAP. Compared to the contralateral control (panel a), heavy GFAP staining is found at the border or infarct (panel e) following MCAO in vehicle-treated animals. However, when rats were treated with NRG1β, GFAP expression was dramatically reduced in the peri-infarct regions (panel f). * denotes infarct core or the corresponding region in the contralateral control; # denotes non-ischemic tissues or the corresponding region in the contralateral control. Scale bar is 100 μM.

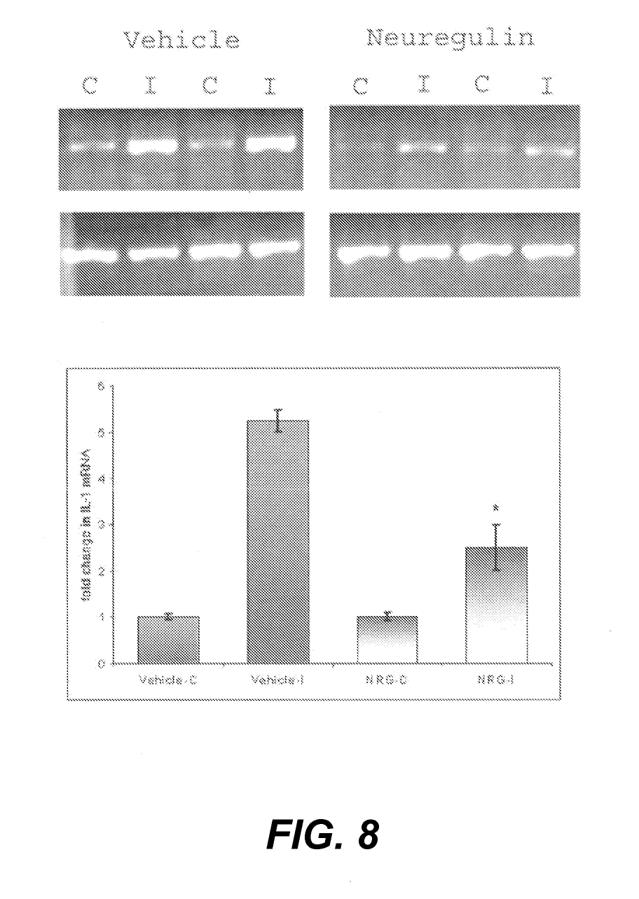

FIG. 8 is a composite of pictures and graphs showing that NRG1β reduces MCAO/reperfusion-induced IL-1β mRNA levels. Rats were treated with NRG1β or vehicle then subjected to MCAO. RNA was isolated and IL-1 β mRNA expression was measured by RT-PCR. The expression of IL-1 (panel a) and GAPDH (panel b) mRNA is shown (n=4 for each condition). Panel c shows the average percentage of change±SEM in IL-1 mRNA levels from NRG-β-treated rat compared to vehicle-treated controls after normalization to GAPDH (* denotes P<0.05). I=ipsilateral hemisphere; C=contralateral hemisphere.

### DETAILED DESCRIPTION

The file of this patent contains at least one drawing executed in color. Copies of this patent with color drawings will be provided by the Patent and Trademark Office upon request and payment of the necessary fee.

The practice of the present invention will employ, unless otherwise indicated, conventional methods of molecular biology, cell biology, Neurology, biochemistry and microbiology within the skill of the art. Such techniques are explained fully in the literature. All publications, patents and patent applications cited herein, whether supra or infra, are hereby incorporated by reference in their entirety.

One aspect of the present invention relates to a method for preventing or ameliorating secondary neuronal injury and inflammation following traumatic brain injury (TBI). The method comprises the step of administering into a subject in need of such treatment an effective amount of a pharmaceutical composition containing a NRG, a variant of NRG, or an expression vector encoding a NRG or a variant of NRG.

### NRG and NRG Variant

The term “neuregulin (NRG),” as used herein, refers to a family of proteins, including NRG1 (Entrez GeneID 3804), NRG2 (Entrez GeneID 9542), NRG3 (Entrez GeneID 10718) and NRG4 (Entrez GeneID 145957), that are involved in the development of the nervous system. The term “NRG1,” as used herein, also includes all NRG-1 isoforms, including acetylcholine receptor inducing activity (ARIA), glial growth factor (GGF), heregulin and neu differentiation factor (NDF) [Falls, D. L., et al., Cell, 1993. 72(5): p. 801-15; Wen, D., et al., Cell, 1992. 69(3): p. 559-72.] NRG-1 isoforms are synthesized as transmembrane precursors consisting of either an immunoglobulin-like or cysteine-rich domain, an EGF-like domain, a transmembrane domain and a cytoplasmic tail [Fischbach, et al., Annu Rev Neurosci, 1997. 20: p. 429-58, 18, 22; Falls, D. L., Exp Cell Res, 2003. 284(1): p. 14-30; Talmage, D. A., et al., J Comp Neurol, 2004. 472(2): p. 134-9.] NRG-1 isoforms are generated from one gene by alternative mRNA splicing, and most of them are synthesized as part of a larger transmembrane precursor. The two major classes of NRG-1 include α and β isoforms. The NRG-1β isoforms predominate in the nervous system, while .alpha. isoforms are prevalent in mesenchymal cells. The β isoforms are 100 to 1,000 fold more potent in stimulating AChR synthesis in skeletal muscle and Schwann cell proliferation [Buonanno, A., et al., Curr Opin Neurobiol, 2001. 11(3): p. 287-96.] The effects of NRG-1 appear to be mediated by interaction with a class of tyrosine kinase receptors related to the epidermal growth factor (EGF) receptor which includes erbB2, erbB3 and erbB4 [Burden, S., et al., Neuron, 1997. 18(6): p. 847-55.]. The EGF-like domain of NRG-1 appears to be sufficient for activation of erbB receptors and downstream signal transduction pathways [Holmes, W. E., et al., Science, 1992. 256(5060): p. 1205-10.]. NRG-1 stimulates the tyrosine phosphorylation of these receptors and the subsequent activation of various signal transduction mechanisms including Map kinase, P13 kinase and CDK5 [Fu, A. K., et al., Nat Neurosci, 2001. 4(4): p. 374-81; Alroy, I., et al., FEBS Lett, 1997. 410(1): p. 83-6.

Neuregulin 2 (NRG2) is a novel member of the neuregulin family of growth and differentiation factors. Through interaction with the ErbB family of receptors, NRG2 induces the growth and differentiation of epithelial, neuronal, glial, and other types of cells. The gene consists of 12 exons and the genomic structure is similar to that of neuregulin 1 (NRG1). NRG1 and NRG2 mediate distinct biological processes by acting at different sites in tissues and eliciting different biological responses in cells. The NRG2 gene is located close to the region for demyelinating Charcot-Marie-Tooth disease locus, but is not responsible for this disease. Alternative transcripts encoding distinct isoforms have been described. (Chang H et al. Nature (1997) 387: 509-12; Carraway K L et al. (1997) Nature 387: 512-6).

Neuregulin 3 (NRG3) binds to the extracellular domain of the ERBB4 receptor tyrosine kinase but not to the related family members ERBB2 or ERBB3. NRG3 binding stimulates tyrosine phosphorylation of ERBB4. Variants of the NRG3 gene have been linked to a susceptibility to schizophrenia (Zhang D, et al. Proc. Natl. Acad. Sci. U.S.A. (1997) 94: 9562-7; Chen P L et al. Am. J Hum. Genet. (2009) 84: 21-34).

Neuregulin 4 (NRG4) activates type-1 growth factor receptors (EGFR) to initiating cell-to-cell signaling through tyrosine phosphorylation. Loss of expression of NRG4 is frequently seen in advanced bladder cancer while increased NRG4 expression correlates to better survival (Haran D et al. Oncogene (1999) 18: 2681-9; Memon A A et al., Br. J Cancer (2004) 91: 2034-41).

As used herein, a “variant of a NRG” is a polypeptide that differs from a native NRG polypeptide in one or more substitutions, deletions, additions and/or insertions, such that the bioactivity or immunogenicity of the native NRG polypeptide is not substantially diminished. In other words, the bioactivity of a variant NRG polypeptide may be enhanced or diminished by less than 50%, and preferably less than 20%, relative to the native NRG polypeptide. Variant NRG polypeptides include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Modifications and changes can be made in the structure of a NRG polypeptide and still obtain a molecule having biological activity and/or immunogenic properties. Because it is the interactive capacity and nature of a NRG polypeptide that defines that polypeptide's biological activity, certain amino acid sequence substitutions can be made in a NRG polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art. It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those that are within +/−1 are particularly preferred, and those within +/−0.5 are even more particularly preferred.

Substitution of like amino acids can also be made on the basis of hydrophilicity, particularly where the biological functional equivalent polypeptide or polypeptide fragment, is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of a NRG as set forth above.

TABLE 1
Amino Acid Substitutions

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

A NRG variant may also, or alternatively, contain nonconservative changes. In a preferred embodiment, variant NRG polypeptides differ from a native NRG sequence by substitution, deletion or addition of five amino acids or fewer. NRG variants may also (or alternatively) be modified by, for example, the deletion or addition of amino acids that have minimal influence on the immunogenicity, secondary structure, tertiary structure, and hydropathic nature of the native NRG polypeptide.

NRG variants preferably exhibit at least about 70%, more preferably at least about 90% and most preferably at least about 95% sequence homology to the original NRG polypeptide.

NRG variant also includes a polypeptide that is modified from the original NRG polypeptide by either natural process, such as post-translational processing, or by chemical modification techniques which are well known in the art. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a fluorophore or a chromophore, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The term “NRG variant” also includes fusion proteins containing a NRG-related polypeptide and a non-NRG-related polypeptide. Within a fusion protein, the NRG-related polypeptide can correspond to all or a portion of a NRG. In a preferred embodiment, a fusion NRG comprises at least one biologically active portion of a NRG.

As used herein, a “biologically active portion” of a NRG includes a fragment of a NRG comprising amino acid sequences sufficiently homologous to or derived from the amino acid sequence of the NRG, which includes fewer amino acids than the full length NRG, and exhibits at least one biological activity of the NRG. Typically, a biologically active portion of a NRG comprises a domain or motif with at least one activity of the NRG. A biologically active portion of a NRG can be a polypeptide, which is, for example, 10, 25, 50, 100, 200 or more amino acids in length.

Within the fusion protein, the NRG-related polypeptide and the non-NRG-related polypeptide are fused in-frame to each other. The non-NRG-related polypeptide can be fused to the N-terminus or C-terminus of the NRG-related polypeptide. A peptide linker sequence may be employed to separate the NRG-related polypeptide from non-NRG-related polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the NRG-related polypeptide and non-NRG-related polypeptide; and (3) the lack of hydrophobic or charged residues that might react with the polypeptide functional epitopes. Preferred peptide linker sequences contain gly, asn and ser residues. Other near neutral amino acids, such as thr and ala may also be used in the linker sequence. Amino acid sequences which may be used as linkers are well known in the art. The linker sequence may generally be from 1 to about 50 amino acids in length. Linker sequences are not required when the NRG-related polypeptide and non-NRG-related polypeptide have non-essential N-terminal amino acid regions that can be used to separate the functional domains and prevent steric interference.

### Expression Vectors

The expression vectors of the present invention include plasmid vectors and viral vectors. The plasmid vectors typically include a circular double-stranded DNA loop into which additional DNA segments can be ligated. The plasmid vectors of the invention comprise one or more regulatory sequences operably linked to a polynucleotide encoding a NRG or NRG variant in a form suitable for expression of the polynucleotide in a target cell.

As used herein, the term “regulatory sequences” refers to DNA sequences necessary for the expression of an operably linked coding sequence in a particular host organism. The term “regulatory sequence” is intended to include promoters, enhancers and other expression control elements (e.g., polyadenylation signals). Regulatory sequences include those which direct constitutive expression of a nucleotide sequence in many types of host cells and those which direct expression of the nucleotide sequence only in certain host cells (e.g., tissue-specific regulatory sequences).

A nucleic acid sequence is “operably linked” to another nucleic acid sequence when the former is placed into a functional relationship with the latter. For example, a DNA for a presequence or secretory leader peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice.

It will be appreciated by those skilled in the art that the design of the expression vector can depend on such factors as the type of target cell, the level of expression of protein desired, and the like. The expression vectors of the invention can be introduced into the target cells to thereby produce proteins or peptides, such as NRGs and NRG variants.

In one embodiment, the expression vector of the invention is a mammalian expression vector. Examples of mammalian expression vectors include pCDM8 and pMT2PC. When used in mammalian cells, the expression vector's control functions are often provided by viral regulatory elements. For example, commonly used promoters are derived from polyoma, adenovirus 2, cytomegalovirus and Simian Virus 40.

In another embodiment, the mammalian expression vector is capable of directing expression of the polynucleotide preferentially in a particular cell type (e.g., neurons) using tissue-specific regulatory elements. Examples of neuron-specific promoters (e.g., the neurofilament promoter)

A number of methods have been developed to deliver the plasmid vector to the target cells. Examples of the delivery methods include, but are not limited to, liposomes-mediated gene transfer, polycationic condensed DNA linked or unlinked to killed adenovirus, ligand linked DNA, eukaryotic cell delivery vehicles cells, deposition of photopolymerized hydrogel materials, handheld gene transfer particle gun, ionizing radiation, nucleic charge neutralization or fusion with cell membranes.

The viral vectors include, but are not limited to, retroviral vector, lentiviral vectors, adenoviral vectors, adeno-associated viral (AAV) vectors, herpes viral vectors, and alphavirus vectors. The viral vector can also be an astrovirus, coronavirus, orthomyxovirus, papovavirus, paramyxovirus, parvovirus, picornavirus, poxvirus, or togavirus viral vector.

In certain embodiment, a regulatable expression system is employed to control the level and duration of NRG expression from an expression vector. These systems are briefly described below:

Tet-on/off system. The Tet-system is based on two regulatory elements derived from the tetracycline-resistance operon of the E. coli Tn10 transposon: the tet repressor protein (TetR) and the Tet operator DNA sequence (tetO) to which TetR binds. The system consists of two components, a “regulator” and a “reporter” plasmid. The “regulator” plasmid encodes a hybrid protein containing a mutated Tet repressor (rtetR) fused to the VP 16 activation domain of herpes simplex virus. The “reporter” plasmid contains a tet-responsive element (TRE), which controls the “reporter” gene of choice. The rtetR-VP 16 fusion protein can only bind to the TRE, therefore activates the transcription of the “reporter” gene, in the presence of tetracycline. The system has been incorporated into a number of viral vectors including retrovirus, adenovirus and AAV.

Ecdysone system. The ecdysone system is based on the molting induction system found in Drosophila, but modified for inducible expression in mammalian cells. The system uses an analog of the drosophila steroid hormone ecdysone, muristerone A, to activate expression of the gene of interest via a heterodimeric nuclear receptor. Expression levels have been reported to exceed 200-fold over basal levels with no effect on mammalian cell physiology.

Progesterone system. The progesterone receptor is normally stimulated to bind to a specific DNA sequence and to activate transcription through an interaction with its hormone ligand. Conversely, the progesterone antagonist mifepristone (RU486) is able to block hormone induced nuclear transport and subsequent DNA binding. A mutant form of the progesterone receptor that can be stimulated to bind through an interaction with RU486 has been generated. To generate a specific, regulatable transcription factor, the RU486-binding domain of the progesterone receptor has been fused to the DNA-binding domain of the yeast transcription factor GAL4 and the transactivation domain of the HSV protein VP16. The chimeric factor is inactive in the absence of RU486. The addition of hormone, however, induces a conformational change in the chimeric protein, and this change allows binding to a GAL4-binding site and the activation of transcription from promoters containing the GAL4-binding site.

Rapamycin system. Immunosuppressive agents, such as FK506 and rapamycin, act by binding to specific cellular proteins and facilitating their dimerization. For example, the binding of rapamycin to FK506-binding protein (FKBP) results in its heterodimerization with another rapamycin binding protein FRAP, which can be reversed by removal of the drug. The ability to bring two proteins together by addition of a drug potentiates the regulation of a number of biological processes, including transcription. A chimeric DNA-binding domain has been fused to the FKBP, which enables binding of the fusion protein to a specific DNA-binding sequence. A transcriptional activation domain also has been fused to FRAP. When these two fusion proteins are co-expressed in the same cell, a fully functional transcription factor can be formed by heterodimerization mediated by addition of rapamycin. The dimerized chimeric transcription factor can then bind to a synthetic promoter sequence containing copies of the synthetic DNA-binding sequence. This system has been successfully integrated into adenoviral and AAV vectors. Long-term regulatable gene expression has been achieved in both mice and baboons.

The plasmid and viral expression vectors may be used to provide long-term in vivo expression (weeks and even months) of NRG or NRG variant.

### Pharmaceutical Compositions

The pharmaceutical composition of the present invention contains one or more NRGs, NRG variants, expression vectors encoding NRGs or NRG variants, or combinations thereof. In certain embodiments, the pharmaceutical composition further contains a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The another aspect of the invention includes methods for preparing pharmaceutical compositions for modulating the expression or activity of a polypeptide or polynucleotide corresponding to an NRG or NRG variant of the invention. Such methods comprise formulating a pharmaceutically acceptable carrier with an agent which modulates expression or activity of a NRG or NRG variant. Such compositions can further include additional active agents. Thus, the invention further includes methods for preparing a pharmaceutical composition by formulating a pharmaceutically acceptable carrier with an agent, which modulates expression or activity of a NRG or NRG variant and one or more additional bioactive agents.

A pharmaceutical composition of the invention is formulated to be compatible with its intended route of administration. Examples of routes of administration include parenteral, e.g., intravenous, intraarterial, intramuscular, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NRG or NRG variant) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form, as used herein, includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of the active ingredient (e.g., a NRG or NRG variant) in the pharmaceutical composition can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Active ingredients which exhibit large therapeutic indices are preferred. While active ingredients that exhibit toxic side effects may be used, care should be taken to design a delivery system that targets such active ingredients to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The plasmid and viral vectors of the present invention can be delivered to a subject by, for example, intravenous administration, intraportal administration, intrabiliary administration, intra-arterial administration, direct injection into the liver parenchyma, by intramuscular injection, by inhalation, by perfusion, or by stereotactic injection. The pharmaceutical preparation of the plasmid and viral vectors can include an acceptable diluent, or can comprise a slow release matrix in which the plasmid and viral vectors are imbedded. Alternatively, where the viral vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

In a preferred embodiment, the method for preventing or ameliorating secondary neuronal injury and inflammation following TBI comprises administering into a subject in need of such treatment an effective amount of a pharmaceutical composition containing NRG1. In one embodiment, NRG1 is administered within 24 hours of the TBI. In another embodiment, NRG1 is administered intravascularly or intramuscularly at doses between 0.05-5000 ug/kg body weight; preferably, 0.5-1000 ug/kg, more preferably, 1-500 ug/kg, most preferably, 5-100 ug/kg. In another embodiment, NRG1 is administered daily for a period of 3-14 days.

In another embodiment, the method comprises administering into the subject an effective amount of a pharmaceutical composition containing (1) a NRG or a variant of NRG, and (2) an expression vector encoding a NRG or a variant of NRG. The NRG or variant of NRG provides short-term effect in preventing or ameliorating secondary neuronal injury and inflammation following TBI. The expression vector expresses the NRG or variant of NRG in vivo and provides long-term effect in preventing or ameliorating secondary neuronal injury and inflammation following TBI. The NRG and NRG expressing vector may be injected concurrently or separately.

Another aspect of the present invention relates to a method for preventing or treating acute CNS injuries. The method comprises the step of administering into a subject in need of such treatment an effective amount of a pharmaceutical composition containing a NRG, a variant of NRG, or an expression vector encoding a NRG or a variant of NRG.

In a preferred embodiment, the method for preventing or treating acute CNS injuries comprises the step of administering into a subject in need of such treatment an effective amount of a pharmaceutical composition containing NRG1. In one embodiment, NRG1 is administered within 24 hours of the acute CNS injury. In another embodiment, NRG1 is administered intravascularly or intramuscularly at doses between 0.05-5000 ug/kg body weight; preferably, 0.5-1000 ug/kg, more preferably, 1-500 ug/kg, most preferably, 5-100 ug/kg. In another embodiment, NRG1 is administered daily for a period of 3-14 days.

### Kits

The invention also encompasses kits for preventing and treating acute CNS injuries, and kits for preventing or amelioratring secondary neuronal injury and inflammation following traumatic brain injury (TBI). The kits comprise one or more effective doses of a NRG, an NRG variant, an expression vector encoding a NRG or NRG variant, or combinations thereof along with a label or labeling with instructions on using the NRG, NRG variant, or expression vector to prevent or ameliorate secondary neuronal injury and inflammation following TBI according to the methods of the invention. In certain embodiments, the kits can comprise components useful for carrying out the methods such as devices for delivering the NRG, NRG variant, or expression vector. In certain embodiments, the kit can comprise components useful for the safe disposal of devices for delivering the NRG, NRG variant, or expression vector, e.g., a sharps container for used syringes.

In one embodiment, the NRG, NRG variant, or expression vector in the kit is formulated for intravascular administration. In another embodiment, the NRG, NRG variant, or expression vector in the kit is formulated for intramuscular administration. In another embodiment, the NRG, NRG variant, or expression vector in the kit is formulated for subcutaneous administration. The NRG, NRG variant, or expression vector may be formulated for slow release. In one embodiment, the NRG, NRG variant, or expression vector is embedded in an implantable inert matrix.

The present invention is further illustrated by the following examples which should not be construed as limiting. The contents of all references, patents and published patent applications cited throughout this application, as well as the Figures and Tables are incorporated herein by reference.

### Example 1

### ErbB4 Receptors are Expressed Apoptotic and Degenerating Neurons

After erB4 immunohistochemistry, brain sections harvested from rats subjected to middle cerebral artery occlusion (MCAO) were stained with Fluoro-Jade, a marker for degenerating neurons, and with antibodies against erbB4. As shown in FIG. 1, many neurons in the cortex were Fluoro-Jade-positive (panel A; green). ErbB4 positive cells (panel B; red) were co-localized in Fluoro-Jade-positive neurons (panel C; yellow). Similarly, TUNEL staining (panel D; green) and erbB4 (panel E; red) were double-labeled (panel F; yellow) in a subpopulation of cells in the ipsilateral brain.

### Example 2

### ErbB4 Expression is Upregulated in Macrophages/Microglia but not in Astrocytes Following MCAO

Sections from the ipsilateral hemisphere of rats subjected to MCAO were double labeled with antibodies against erbB4 (FIG. 2, panel A) and glial fibrillary acidic protein (GFAP) (FIG. 2, panel B). Cells in the peri-infarct regions did not show co-localization of erbB4 and GFAP (FIG. 2, panel C). Co-localization of erbB4 (green) and Mac-1/CD11b (red) indicated that erbB4 is found in a subset of macrophages/microglia (FIG. 2, panel D).

### Example 3

### NRG-1β Treatment Reduces MCAO/Reperfusion-Induced Brain Infarction

FIG. 3 shows representative TTC stained brain sections from rats injected with vehicle (panel a; n=11), NRG1 β (panel b; n=7) or NRG1α (panel c; n=3) before MCAO. NRG1β (2.5 μg/kg) or NRG1α (2.5 μg/kg) was given by a single intra-arterial injection immediately before MCAO. Adult male Sprague-Dawley rats weighing 250-300 g were used for this study. A total of 164 rats were used in this study. Rats were anesthetized with a ketamine/xylazine solution (10 mg/kg, IP) and subjected to left MCAO. MCAO was induced by the intraluminal suture method where the left common carotid artery (CCA) was exposed through a midline incision and was carefully dissected free from surrounding nerves and fascia. The occipital artery branches of the external carotid artery (ECA) were then isolated, and the occipital artery and superior thyroid artery branches of the ECA were coagulated. The ECA was dissected further distally. The internal carotid artery (ICA) was isolated and carefully separated from the adjacent vagus nerve, and the pterygopalatine artery was ligated close to its origin with a 6-0 silk suture. Then, a 40 mm 3-0 surgical monofilament nylon suture (Harvard Apparatus, Holliston, Mass.) was coated with poly-L-lysine with its tip rounded by heating near a flame. The filament was inserted from the ECA into the ICA and then into the circle of Willis to occlude the origin of the left MCA. The suture was inserted 18 to 20 mm from the bifurcation of the CCA to occlude the MCA. After 1.5 hour of ischemia, the nylon suture was withdrawn and the ischemic brain tissue was reperfused for 24 hours before sacrificing. Core body temperature was monitored with a rectal probe and maintained at 37° C. with a Homeothermic Blanket Control Unit (Harvard Apparatus) during anesthesia. To determine the effects of NRG-1 on ischemic stroke, rat were injected intravascularly with a single bolus 10 μl dose of vehicle (1% BSA in PBS) or NRG-1β (1-50 umol/L NRG-1 (EGF-like domain, R&D Systems, Minneapolis, Minn.) dissolved in 1% BSA/PBS) through a Hamilton syringe at a rate of 5 μl/min. This resulted in the administration of 0.5-2.5 μg of NRG-1/kg body weight. NRG-1 or vehicle was administered by bolus injection into the ICA through ECA. Solutions were administered either before MCAO or immediately following 1.5 hours of MCAO and either 0, 4 or 12 hours of reperfusion. Animals were sacrificed 24 hours after reperfusion or after 14 days for the long-term studies. Animals were killed 24 hours later and the brains were sliced into 2 mm sections and stained with 2,3,5-triphenyltetrazolium chloride (TTC). Infarct volumes in brains from vehicle and NRG1 treated animals are shown in the graph (panel d). The data demonstrate that NRG1β treatment reduces MCAO/reperfusion-induced brain infarction.

### Example 4

### NRG-1β Suppresses MCAO/Reperfusion-Induced Apoptotic Damage in Rat Brain

Rats were subjected to MCAO for 1.5 hours followed by reperfusion for 24 hours. FIG. 4 shows representative views of TUNEL labeling of rat brain sections (n=5 for each condition). TUNEL assay was performed with a DeadEND Fluorometric TUNEL System (Promega, Madison, Wis.) according to the manufacturer's instructions. Slides were then washed with PBS and mounted with Vectashield Mounting Medium containing DAPI. All sections were examined by fluorescence microscopy in three random middle cerebral artery served areas in the inner border of the infarct in the ischemic front-parietal cortex of each rat. In animals given vehicle or neuregulin-1, cortex and striatum were examined in three or more 20 μm sections per animal. TUNEL staining was found in the cortex (panel a) and striatum (panel b) following MCAO while no TUNEL staining was seen in the cortex (panel c) and reduced levels were seen in the striatum (panel d) in NRG1β-treated rats. The coronal brain image (˜bregma+1.2 mm) indicates the areas observed in the sections (panel e).

### Example 5

### NRG-1 Treatment Reduces MCAO/Reperfusion-Induced Brain Infarction

FIG. 5 shows representative TTC stained coronal brain sections from rats injected with vehicle (panel a) or NRG1 immediately after MCAO (panel b) and 4 hours after reperfusion (panel c). Infarct volumes in brains from rats treated with vehicle (n=10) or NRG1 immediately after MCAO (R0; n=8), 4 hours after reperfusion (R4; n=6) or 12 hours after reperfusion (R12; n=8) are show in the graph (panel d). The time line (panel e) illustrates the MCAO protocol and NRG1 injections.

### Example 6

### NRG-1 Administration Resulted in a Significant Improvement in Neurological Outcome

NRG1 was administered after MCAO and 4 hours of reperfusion. As shown in FIG. 6, neurological function was graded on a scale of 0-4 (normal score 0, maximal deficit score 4). All animals were tested prior to surgery (controls; n=14) and after treatment with NRG-1 or vehicle. The NRG1 treated group (n=9) displayed a 33% improvement in neurological score compared with vehicle treated rats (n=5).

### Example 7

### NRG1β Prevents Microglial and Astrocytic Activation Following MCAO

Rats were subjected to MCAO followed by reperfusion for 24 hours (n=5 for each condition). NRG1β or vehicle was injected intraarterially as described above. Sections were labeled for immunohistochemistry with an antibody against ED-1. As shown in FIG. 7, while no staining was seen in the contralateral side (panel a), ED-1 labeled cells are present in the ipsilateral hemisphere (panel b) following MCAO in vehicle-treated animals. Few ED-1 positive cells are found in animals treated with NRG1β (panel c). To assess astrocytic activation, sections were labeled for immunohistochemistry with an antibody against GFAP. Compared to the contralateral control (panel a), heavy GFAP staining is found at the border or infarct (panel e) following MCAO in vehicle-treated animals. However, when rats were treated with NRG-1β, GFAP expression was dramatically reduced in the peri-infarct regions (panel f).

### Example 8

### NRG1β Prevents Microglial and Astrocytic Activation Following MCAO

Rats were treated with NRG1β or vehicle then subjected to MCAO. RNA was isolated and IL1β mRNA expression was measured by RT-PCR. FIG. 8 shows the mRNA expression levels of IL-1 (panel a) and GAPDH (panel b) (n=4 for each condition). Panel c shows the average percentage of change±SEM in IL-1 mRNA levels from NRG1β-treated rat compared to vehicle-treated controls after normalization to GAPDH.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method for preventing or ameliorating secondary neuronal injury and inflammation following traumatic brain injury (TBI), comprising: administering into a mammal in need of such treatment an effective amount of a pharmaceutical composition comprising a neuregulin (NRG), a variant of NRG, or an expression vector encoding a NRG or a variant of NRG.

2. The method of claim 1, wherein said pharmaceutical composition comprises NRG1 or a variant of NRG1.

3. The method of claim 2, wherein said NRG1 or a variant of NRG1 is administered intra-vascularly at a dose between 0.05-5000 μg/kg body weight.

4. The method of claim 3, wherein said NRG1 or a variant of NRG1 is administered intra-vascularly at a dose between 0.5-1000 μg/kg body weight.

5. The method of claim 2, wherein said pharmaceutical composition comprises NRG1β or a variant of NRG1β.

6. The method of claim 2, wherein said pharmaceutical composition comprises NRG1α or a variant of NRG1α.

7. The method of claim 1, wherein said pharmaceutical composition is administered by intra-vascular injection.

8. The method of claim 7, wherein said pharmaceutical composition is administered by intra-arterial injection.

9. The method of claim 1, wherein said pharmaceutical composition is administered within 24 hours of trauma.

10. The method of claim 1, comprising: administering into the subject an effective amount of a pharmaceutical composition comprising: (1) a neuregulin (NRG) or a variant of NRG; and (2) an expression vector encoding a NRG or a variant of NRG.

11. A method for treating acute CNS injuries, comprising: administering into a subject in need of such treatment an effective amount of a pharmaceutical composition comprising a neuregulin (NRG), a variant of NRG, or an expression vector capable of in vivo expression of a NRG or a variant of NRG.

12. The method of claim 11, wherein said pharmaceutical composition comprises NRG1 or a variant of NRG1.

13. The method of claim 12, wherein said NRG1 or a variant of NRG1 is administered intra-vascularly at a dose between 0.05-5000 μg/kg body weight.

14. The method of claim 13, wherein said NRG1 or a variant of NRG1 is administered intra-vascularly at a dose between 0.5-1000 μg/kg body weight.

15. The method of claim 12, wherein said pharmaceutical composition comprises NRG1β or a variant of NRG1β.

16. The method of claim 11, wherein said pharmaceutical composition is administered by intra-vascular injection.

17. The method of claim 16, wherein said pharmaceutical composition is administered by intra-arterial injection.

18. The method of claim 16, wherein said pharmaceutical composition is administered by intra-arterial injection.

19. The method of claim 11, wherein said pharmaceutical composition comprises a NRG and an expression vector encoding a NRG.

20. A kit for preventing or ameliorating secondary neuronal injury and inflammation following traumatic brain injury (TBI), comprising: (1) a NRG, a variant of NRG, or an expression vector encoding a NRG or a variant of NRG; and (2) an instruction on how to use the NRG, the variant of NRG, or the expression vector encoding a NRG or a variant of NRG.
